(19) United States
(12) Patent Application Publication
(10) Pub. No.: US 2019/0233891 A1
(43) Pub. Date: Aug. 1, 2019

(54) FOR HUMAN LEUKOCYTE ANTIGEN GENOTYPING METHOD AND DETERMINING HLA HAPLOTYPE DIVERSITY IN A SAMPLE POPULATION

(71) Applicant: Sirona Genomics, Inc., Mountain View, CA (US)

(72) Inventors: Chunlin WANG, Mountain View, CA (US); Ming LI, Mountain View, CA (US)

(73) Assignee: Sirona Genomics, Inc., Mountain View, CA (US)

(21) Appl. No.: 16/335,733

(22) PCT Filed: Sep. 26, 2017
(86) PCT No.: PCT/US2017/053464

§ 371 Date: Mar. 22, 2019

Related U.S. Application Data
(60) Provisional application No. 62/399,707, filed on Sep. 26, 2016.

(51) Int. Cl. C12Q 1/6881; G16B 20/00; G16B 20/20

(52) U.S. Cl. CPC C12Q 1/6881; G16B 20/20; G16B 20/00

(57) ABSTRACT

A method for determining the association of Human Leukocyte Antigen (HLA) alleles at adjacent loci in genomic DNA from a biological sample obtained from a human subject is disclosed. A method and system of validating correctness of an assignment of an allele variant to a genetic human leucocyte antigen (HLA) locus are also disclosed.

### FIELD

The present disclosure generally relates to determining the haplotype of an individual from genomic sequence information.

### BACKGROUND

Next generation sequencing technology (NGS) allows genotyping for Human Leukocyte Antigen (HLA) genes with full resolution for multiple samples in a single run. Compared to traditional methods of Sequence-Specific Oligonucleotide (SSO), sequence-Specific Primer (SSP) and Sanger sequencing, NGS delivers more complete coverage of the genome and phased contig sequences to resolve ambiguities. Despite the advantages of the NGS technology, validation of the HLA typing results is challenging because present technologies provide only up to 3 field resolution.

The present disclosure provides high throughput typing method can generate accurate genotype calls for all 11 major HLA genes with 4 field resolution based on NGS technology, satisfying a need in the art for increased accuracy and the ability to rapidly process large numbers of samples in a cost-effective manner.

### SUMMARY

A first aspect of the present disclosure relates to a method for determining the association of Human Leukocyte Antigen (HLA) alleles at adjacent loci in genomic DNA from a biological sample obtained from a human subject. The method comprises the steps of amplifying genomic DNA from the sample by long-range PCR reaction; sequencing the amplified DNA; determining the association frequency of the genotype of an allele of a first locus with the genotype of an allele of at least one adjacent locus by reference to a database of loci associations; and reporting an association score of said allele in a first locus with the genotype of the allele in at least one adjacent locus, thereby determining the association of Human Leukocyte Antigen (HLA) alleles at adjacent loci in genomic DNA.

A second aspect of the present disclosure relates to a database matrix for use in the analysis of association score of an allele to be assigned to a first locus and at least one additional locus. The database matrix comprises a field corresponding to an allele to be genotyped; at least one other field corresponding to another allele at a different locus; at least another field corresponding to the probability of the allele to be genotyped to a locus and the at least one other field as expressed as a probability.

A third aspect of the present disclosure relates to a method of validating correctness of an assignment of an allele variant to a genetic human leucocyte antigen (HLA) locus. The method comprises acquiring genotype information representing an assignment of an allele variant to a genetic human leucocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

A fourth aspect of the present disclosure relates to a method of operating a computing device comprising at least one processor, the method comprising: executing the at least one processor to acquire genotype information representing an assignment of an allele variant to a genetic human leukocyte antigen (HLA) locus; and when it is determined that a score representing an association of the allele variant with at least one other allele variant of at least one adjacent genetic locus exists using the determined score to generate an indication indicating the likelihood of the correctness of the assignment of the allele variant to the genetic HLA locus; and reporting the indication.

A fifth aspect of the present disclosure relates to a computing system comprising at least one processor configured to assign at least one partial haplotype to a genetic locus by performing the method according to any one of the first through fourth aspects and their embodiments.

A sixth aspect of the present disclosure relates to a system for validating correctness of a genotype of a sample obtained from a subject, the system comprising: at least one processor; a memory communicatively coupled to the processor, the memory having stored thereon computer executable instructions that, when executed by the at least processor, perform a method comprising: acquiring genotype information representing an assignment of an allele variant to a genetic human leukocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

A seventh aspect of the present disclosure relates to at least one non-transitory computer storage device storing computer-executable instructions that, when executed by at least one processor, cause the at least one processor to perform a method comprising: acquiring a genotype information representing an assignment of an allele variant to a genetic human leucocyte antigen (HLA) locus; acquiring a score representing an association of the allele variant with at least one other allele variant of at least one adjacent genetic locus; using the acquired score to generate an indication indicating correctness of the assignment of the allele variant to the genetic HLA locus; and reporting the indication.

An eighth aspect of the present disclosure relates to a method of operating a computing device comprising at least one processor, the method comprising: executing the at least one processor to acquire genotype information representing an assignment of an allele variant to a genetic human leucocyte antigen (HLA) locus; accessing a computer readable storage device storing linkage disequilibrium information to determine whether the linkage disequilibrium information includes a score representing an association of the allele variant with at least one other allele variant of at least one adjacent genetic locus; when it is determined that the linkage disequilibrium information includes the score using the determined score to generate an indication indicating correctness of the assignment of the allele variant to the genetic HLA locus; and reporting the indication.

A ninth aspect of the present disclosure relates to a method of assigning an allele to a genetic locus comprising: amplifying coding and non-coding DNA from a genetic locus from a sample of genomic DNA to produce an amplicon; sequencing the amplicon; identifying at least a first allele variant and a second allele variant of the genetic locus from the amplicon; determining a score representing an association of the first allele variant with at least one other allele variant of at least one adjacent genetic locus; and using the determined score to generate an indication indicating correctness of the assignment of the allele variant to the genetic HLA locus.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 shows an example of a software user interface.

FIG. 2 shows an exemplary phasing analysis delivering contiguous contig sequences across entire genes.

FIG. 3 shows an exemplary summary table for genotyping results.

FIG. 4 shows an exemplary pedigree for family trios.

FIG. 5 shows an exemplary scheme for long-range PCR amplification of HLA genes from human genomic DNA.

FIG. 6 shows a summary of the frequency of HLA Class I alleles in the subject population.

FIGS. 7A and 7B show a summary of the frequency of HLA Class II alleles in the subject population.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

A first aspect of the present disclosure relates to a method for determining the association of Human Leukocyte Antigen (HLA) alleles at adjacent loci in genomic DNA from a biological sample obtained from a human subject. The method comprises the steps of amplifying genomic DNA from the sample by long-range PCR reaction; sequencing the amplified DNA; determining the association frequency of the genotype of an allele of a first locus with the genotype of an allele of at least one adjacent locus by reference to a database of loci associations; and reporting an association score of said allele in a first locus with the genotype of the allele in at least one adjacent locus, thereby determining the association of Human Leukocyte Antigen (HLA) alleles at adjacent loci in genomic DNA.

In some embodiments, the association is determined between three or more loci. In some further embodiments, the two or more loci are in linkage disequilibrium with said first locus.

In other embodiments, the association is determined between four or more loci. In some further embodiments, the three or more loci are in linkage disequilibrium with the first locus.

In still other embodiments, the association is determined between five or more loci. In some further embodiments, the four or more loci are in linkage disequilibrium with the first locus.

In yet other embodiments, the association is determined between 11 loci. In some further embodiments, at least two of said eleven loci are in linkage disequilibrium.

In some embodiments, the first allele is an allele of an HLA locus selected from the group consisting of HLA-A, HLA-DRB1, HLA-DRB3, HLA-DRB4, HLA-DRB5, HLA-DQB1, HLA-DQA1, HLA-DPB1 and HLA-DPA1.

In some embodiments, the database of associations comprises associations of HLA loci for at least 2 loci selected from: HLA-A, HLA-DRB1, HLA-DRB3, HLA-DRB4, HLA-DRB5, HLA-DQB1, HLA-DQA1, HLA-DPB1 and HLA-DPA1.

In some embodiments, the at least one adjacent locus is in linkage disequilibrium with the first locus.

In some embodiments, the method further comprises comparing the association score to a database of association scores associated with a disease. In some further embodiments, the disease is an autoimmune disease.

In other embodiments, the method further comprises comparing the association score to an association score obtained for a different human subject for assessing tissue compatibility.

A second aspect of the present disclosure relates to a database matrix for use in the analysis of association score of an allele to be assigned to a first locus and at least one additional locus. The database matrix comprises a field corresponding to an allele to be genotyped; at least one other field corresponding to another allele at a different locus; at least another field corresponding to the probability of the allele to be genotyped to a locus and the at least one other field as expressed as a probability.

A third aspect of the present disclosure relates to a method of validating correctness of an assignment of an allele variant to a genetic human leucocyte antigen (HLA) locus. The method comprises acquiring genotype information representing an assignment of an allele variant to a genetic human leucocyte antigen (HLA) locus; determining a score representing an association of the allele variant with at least one other allele variant of at least one adjacent genetic locus; using the determined score to generate the indication indicating correctness of the assignment of the allele variant to the genetic HLA locus; and reporting the indication.

In some embodiments, the method further comprises processing a biological sample.

In other embodiments, the method further comprises generating the genotype information.

A fourth aspect of the present disclosure relates to a method of operating a computing device comprising at least one processor, the method comprising executing the at least one processor to: acquire genotype information representing an assignment of an allele variant to a genetic human leukocyte antigen (HLA) locus; and when it is determined that a score representing an association of the allele variant with at least one other allele variant of at least one adjacent genetic locus exists: using the determined score to generate an indication indicating the likelihood of the correctness of the assignment of the allele variant to the genetic HLA locus; and reporting the indication.

In some embodiments, the at least one adjacent genetic locus comprises at least two adjacent genetic loci. In some further embodiments, the at least two adjacent genetic loci are in linkage disequilibrium database with the genetic HLA locus.

In other embodiments, the at least one adjacent genetic locus comprises three or more adjacent genetic loci. In some further embodiments, the at least three adjacent genetic loci are in linkage disequilibrium database with the genetic HLA locus.

In still other embodiments, the at least one adjacent genetic locus comprises four or more adjacent genetic loci. In some further embodiments, wherein the at least four adjacent genetic loci are in linkage disequilibrium database with the genetic HLA locus.

In yet other embodiments, the at least one adjacent genetic locus comprises five or more adjacent genetic loci. In some further embodiments, the at least five adjacent genetic loci are in linkage disequilibrium database with the genetic HLA locus.

In even other embodiments, the at least one adjacent genetic locus is an HLA locus.

In other embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In other embodiments, the method further comprises executing the at least one processor to determine whether the score exists by computing with information, by the at least one processor, a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus. In other further embodiments, the method further comprises, when it is determined that the linkage disequilibrium database does not include the score, generating, by the at least one processor, a second indication indicating that the linkage disequilibrium database does not include the score. In still other further embodiments, the method further comprises, when it is determined that the linkage disequilibrium database does not include the score, flagging, by the at least one processor, at least one of the allele variant and the at least one other allele variant of the at least one adjacent genetic locus. In yet other further embodiments, the method further comprises, when it is determined that the linkage disequilibrium database does not include the score, flagging, by the at least one processor, at least one of the allele variant and the at least one other allele variant of the at least one adjacent genetic locus.

In some embodiments, the indication comprises a numerical value.

In other embodiments, the at least one adjacent genetic locus is in linkage disequilibrium database with the genetic HLA locus.

In some embodiments, reporting the indication comprises displaying a representation of the indication on a display device communicatively coupled with the computing device. In some further embodiments, the representation of the indication is displayed on the display device in a graphical format.

In some embodiments, the method further comprises acquiring second genotype information representing an assignment of the at least one other allele variant to the at least one adjacent genetic locus.

In other embodiments, the method further comprises acquiring the genotype information electronically via a network.

In some embodiments, the genotype information is generated using a genotyping technique that is different from a method of validating correctness of an assignment of an allele variant to a genetic human leucocyte antigen (HLA) locus, that method comprising acquiring genotype information representing an assignment of an allele variant to a genetic human leucocyte antigen (HLA) locus; determining a score representing an association of the allele variant with at least one other allele variant of at least one adjacent genetic locus; using the determined score to generate the indication indicating correctness of the assignment of the allele variant to the genetic HLA locus; and reporting the indication.

In some embodiments, the method further comprises using the indication indicating correctness of the assignment of the allele variant to the genetic HLA locus to assess one or more of the following applications selected from the group consisting of: HLA typing, transplant capability, donor-recipient compatibility and diagnosis of graft versus host disease.

A fifth aspect of the present disclosure relates to a computing system comprising: at least one processor configured to assign at least one partial haplotype to a genetic locus by performing the method according to any one of the first through fourth aspects and their embodiments.

A sixth aspect of the present disclosure relates to a system for validating correctness of a genotype of a sample obtained from a subject, the system comprising: at least one processor; a memory communicatively coupled to the processor, the memory having stored thereon computer executable instructions that, when executed by the at least processor, perform a method comprising: acquiring genotype information representing an assignment of an allele variant to a genetic human leukocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

In some embodiments, the at least one adjacent genetic locus comprises at least two adjacent genetic loci.

In other embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In further some embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, the system further comprises acquiring second genotype information representing an assignment of the at least one other allele variant to the at least one adjacent genetic locus.

In other embodiments, the system further comprises acquiring the genotype information electronically via a network.

In some embodiments, the genotype information is generated using a genotyping technique that is different from a method of validating correctness of an assignment of an allele variant to a genetic human leucocyte antigen (HLA) locus, that method comprising acquiring genotype information representing an assignment of an allele variant to a genetic human leucocyte antigen (HLA) locus; determining a score representing an association of the allele variant with at least one other allele variant of at least one adjacent genetic locus; using the determined score to generate the indication indicating correctness of the assignment of the allele variant to the genetic HLA locus; and reporting the indication.

In other embodiments, reporting the indication comprises displaying a representation of the indication on a display device communicatively coupled with the at least one processor. In some further embodiments, the representation of the indication is displayed on the display device in a graphical format.

A seventh aspect of the present disclosure relates to at least one non-transitory computer storage device storing computer-executable instructions that, when executed by at least one processor, cause the at least one processor to perform a method comprising: acquiring a genotype information representing an assignment of an allele variant to a genetic human leucocyte antigen (HLA) locus; acquiring a score representing an association of the allele variant with at least one other allele variant of at least one adjacent genetic locus; using the acquired score to generate an indication indicating correctness of the assignment of the allele variant to the genetic HLA locus; and reporting the indication.

In some embodiments, the at least one adjacent genetic locus comprises at least two adjacent genetic loci.

In some embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, the method further comprises acquiring second genotype information representing an assignment of the at least one other allele variant to the at least one adjacent genetic locus.

In some embodiments, the method further comprises acquiring the genotype information electronically via a network.

In some embodiments, the genotype information is generated using a genotyping technique that is different from a method comprising the steps of: acquiring genotype information representing an assignment of an allele variant to a genetic human leukocyte antigen (HLA) locus; acquiring second genotype information representing an assignment of the at least one other allele variant to the at least one adjacent genetic locus; determining a score representing an association of the allele variant with at least one other allele variant of at least one adjacent genetic locus; using the determined score to generate an indication indicating correctness of the assignment of the allele variant to the genetic HLA locus; and reporting the indication.

An eighth aspect of the present disclosure relates to a method of operating a computing device comprising at least one processor, the method comprising executing the at least one processor to: acquire genotype information representing an assignment of an allele variant to a genetic human leucocyte antigen (HLA) locus; accessing a computer readable storage device storing linkage disequilibrium information to determine whether the linkage disequilibrium information includes a score representing an association of the allele variant with at least one other allele variant of at least one adjacent genetic locus; when it is determined that the linkage disequilibrium information includes the score: using the determined score to generate an indication indicating correctness of the assignment of the allele variant to the genetic HLA locus; and reporting the indication.

In some embodiments, the method further comprises, when it is determined that the linkage disequilibrium information does not include the score, generating an indication indicating that the linkage disequilibrium information does not include the score. In some further embodiments, the method further comprises, when it is determined that the linkage disequilibrium information does not include the score, flagging at least one of the allele variant and the at least one other allele variant of the at least one adjacent genetic locus.

In some embodiments, reporting the indication comprises displaying a representation of the indication on a display device communicatively coupled with the computing device.

In other embodiments, the representation of the indication is displayed on the display device in a graphical format.

A ninth aspect of the present disclosure relates to a method of assigning an allele to a genetic locus comprising: amplifying coding and non-coding DNA from a genetic locus from a sample of genomic DNA to produce an amplicon; sequencing the amplicon; identifying at least a first allele variant and a second allele variant of the genetic locus from the amplicon; determining a score representing an association of the first allele variant with at least one other allele variant of at least one adjacent genetic locus; and using the determined score to generate an indication indicating correctness of the assignment of the allele variant to the genetic HLA locus.

In some embodiments, the coding and non-coding DNA are from an exon and an adjacent intron.

In other embodiments, the sequencing is done by a next generation sequencing method.

In some embodiments, the coding DNA comprises at least two exons.

In other embodiments, the coding DNA comprises at least three exons.

In still other embodiments, the coding DNA comprises at least four exons.

In some embodiments, the non-coding DNA comprises at least one intron.

In other embodiments, the non-coding DNA comprises at least two introns.

In still other embodiments, the non-coding DNA comprises at least three introns.

In yet other embodiments, the non-coding DNA comprises at least four introns.

In some embodiments, the at least one adjacent genetic locus is in linkage disequilibrium database with the genetic HLA locus.

In some embodiments, the at least one adjacent genetic locus comprises at least two adjacent genetic loci. In some further embodiments, the at least two adjacent genetic loci are in linkage disequilibrium database with the genetic HLA locus.

In other embodiments, the at least one adjacent genetic locus comprises three or more adjacent genetic loci. In some further embodiments, the at least three adjacent genetic loci are in linkage disequilibrium database with the genetic HLA locus.

In still other embodiments, the at least one adjacent genetic locus comprises four or more adjacent genetic loci. In some further embodiments, the at least four adjacent genetic loci are in linkage disequilibrium database with the genetic HLA locus.

In yet other embodiments, the at least one adjacent genetic locus comprises five or more adjacent genetic loci. In some further embodiments, the at least five adjacent genetic loci are in linkage disequilibrium database with the genetic HLA locus.

In some embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

A particular aspect of the present disclosure introduces a HLA typing method based on NGS developed for high throughput applications and then the accuracy is through pedigree analysis on a large cohort of family trios from a disease association study. This high throughput typing method can generate accurate genotype calls for all 11 major HLA genes with 4 field resolution based on NGS technology. For data analysis, three orthogonal algorithms are combined to rank the genotype candidates and generate consensus sequences for individual alleles. Ambiguity is resolved for heterozygous sample by phasing analysis except for certain allele combinations in DPB1. The method can be used to type 96-384 samples in a single sequencing run for high throughput HLA typing applications, including registry typing, disease association and population studies. For family trios, the accuracy of genotyping results at 3 field resolution is assessed through pedigree analysis. Concordance is computed by comparing allele calls of the child to those of the parents. Also we compute the concordance between the automatic and the reviewed calls to assess the accuracy of automatic calls.

Approximately 1500 family trios were typed by this method and the genotype calls made by the software are manually reviewed based on all the quality metrics. The validation based on pedigree analysis provides a way to assess the accuracy of HLA genotyping methods with 3 and 4 field resolution. The high concordance rate between automatic and reviewed calls showed that the software provides high quality calls and the review process can be effortless for trained technicians.

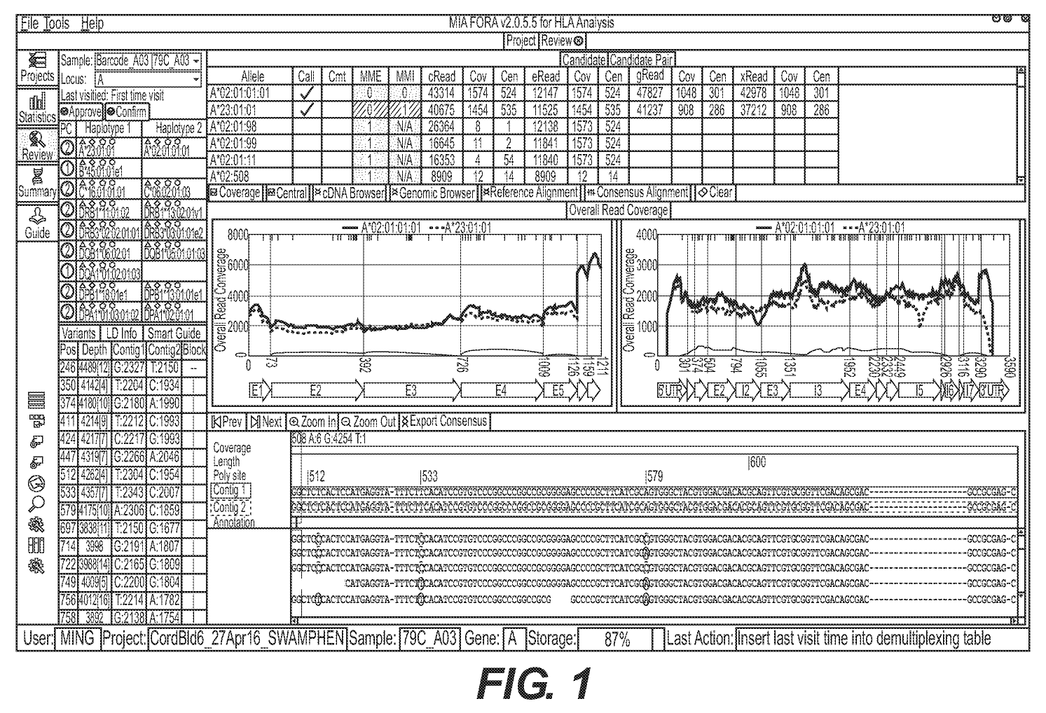

FIG. 1 shows an exemplary display of the Mia For a Software User Interface from Immucor. The software GUI integrates rich information about the mapping statistics and phasing results. Smart Flagging System annotates the genotype calls with confidence score, Common and Well Documented (CWD) alleles and LD information to facilitate manual review process.

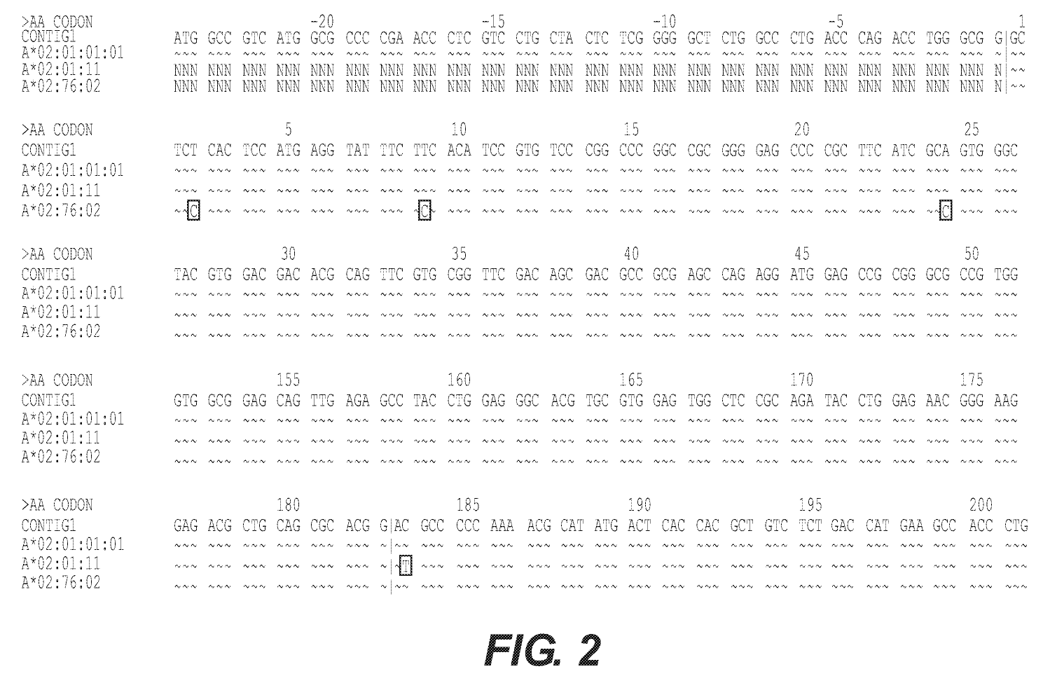

FIG. 2 shows an exemplary alignment between contig and references. Phasing analysis delivers continuous contig sequences across the entire gene and can be aligned to multiple references to find the right match. Mismatches between the contig sequence and references are high-lighted to compare candidate alleles and help to identify novel allele.

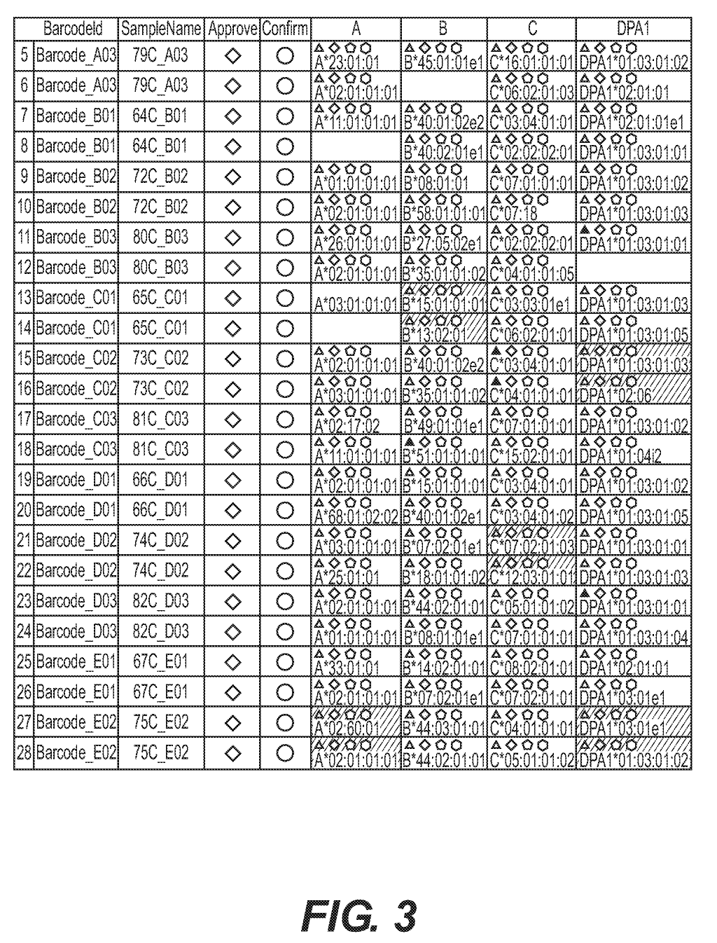

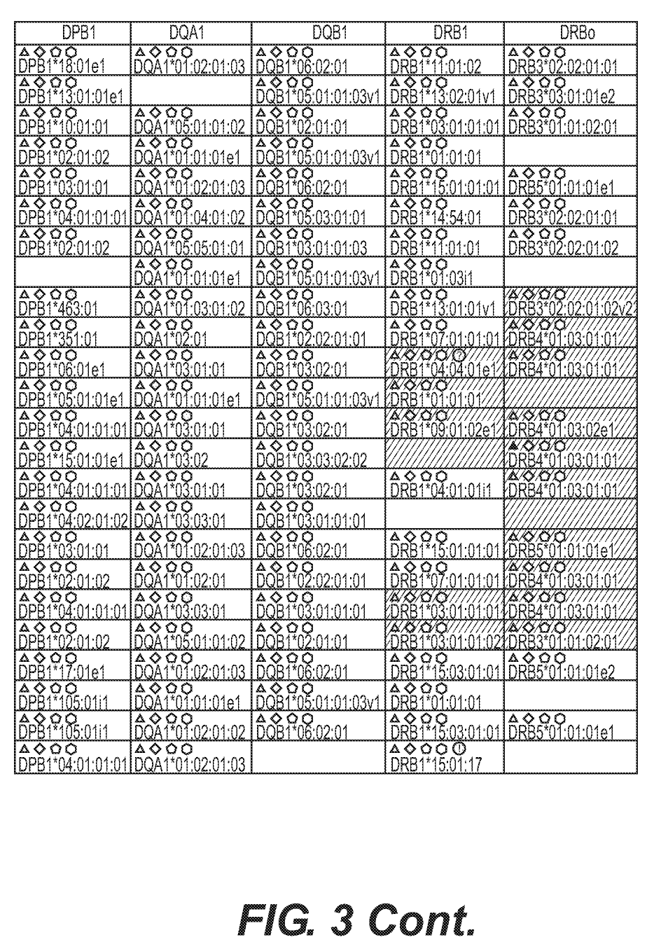

FIG. 3 shows a summary table for genotyping results. The summary table lists the genotype calls for all loci for 96˜384 samples in the project (One sequencing run). Genotype calls are annotated with Smart Flagging system, which incorporate Linkage Disequilibrum (LD) information into the flags. Loci that requires manual review is also high-lighted and review suggestions are provided to user.

### Methods

### Mapping Analysis

All the paired end reads are compared to the reference sequences in the database to find the best matching candidate reference alleles.

### Phasing Analysis

An advanced algorithm is deployed to build a de novo sequence assembly from paired end reads. Two contig sequences are then built from phasing analysis, which has been developed since the Human Genome Project.

### Statistical Modeling

A Bayesian model was developed to characterize the polymorphisms and maximize the likelihood of the matching between the sample and references.

### Reference Database

Internal references were constructed from cloned sequences and combined with IMGT references to produce high quality genotyping calls.

### Linkage Disequilibrium Annotation

Haplotype blocks were flagged in the Smart Flagging to facilitate a manual review process and provide confirmation about the typing results.

### Results

Family trios were used to validate the automatic calls made by the software and also the confirmed calls after manual review. Through pedigree analysis, we assess the accuracy of genotyping results by comparing the allele calls for the child to those of the parents.

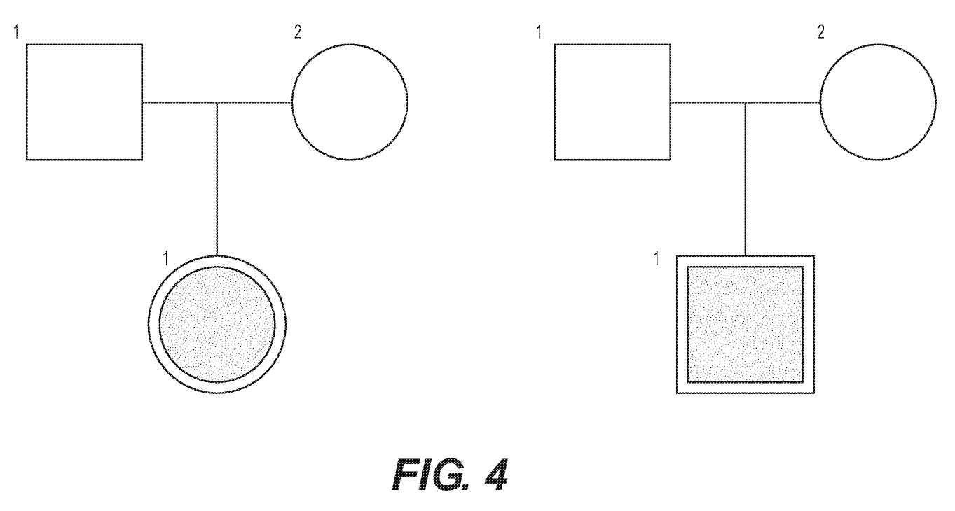

FIG. 4 shows an exemplary pedigree for family trios. Each of the two alleles of the child should match to either the father or the mother. Any violation of this rule was counted as a typing error for the child.

Table 1 lists validated accuracy for major HLA loci at 6 digit resolution. Automatic shows accuracy for calls made by the software. Reviewed shows accuracy for reviewed calls. Concordance compares automatic and reviewed calls, which suggests that less than 1% of the calls require manual review and correction.

TABLE 1
Genotyping accuracy and concordance.

| | Overall | | | | | | | |
|---|---|---|---|---|---|---|---|---|
| | Class I | | | Class II | | | | |
| Locus | HLA-A | HLA-B | HLA-C | DRB1 | DRB3/4/5 | DQA1 | DQB1 | DPA1 |
| Automatic | 99.5% | 98.2% | 99.2% | 95.8% | 98.6% | 99.1% | 99.8% | 100% |
| 98.8% | 99.0% | | | 98.7% | | | | |
| Reviewed | 99.9% | 99.8% | 99.9% | 99.6% | 99.6% | 100% | 100% | 100% |
| 99.8% | 99.86% | | | 99.8% | | | | |
| Concordance* | 99.8% | 99.4% | 99.9% | 97.5% | 99.3% | 99.2% | 99.9% | 100% |

| | | |
|---|---|---|
| 99.4% | 99.7% | 99.2% |

*Concordance between the automatic and reviewed calls including all three members in the family trio.

### CONCLUSIONS

The present disclosure provides a novel system for accurate HLA typing based on NGS technology for high throughput projects with 96-384 samples per run. All 11 major HLA loci can be typed at 8 digit resolution with no ambiguity except for some DPB1 allele combination.

The software delivers validated accuracy above 98.8% for automatic calls at 6 digits. After manual review, the validation rate is above 99.8%.

The overall concordance between automatic and reviewed results is 99.4%.

Combined with Smart Flagging System which incorporate domain knowledge including CWD and LD info, the review process is made effortless for the practitioner.

A second aspect of the present disclosure provides a method for characterizing HLA types. In some embodiments, the method identifies previously uncharacterized linked alleles. In another embodiment, the method identifies transplant compatibility. In another embodiment, the method defines allele associations with, or susceptibility to, disease. In some further embodiments, the disease is an autoimmune disease. In other further embodiments, the disease is an infectious disease.

In an exemplary embodiment, the method was applied to samples from a group of subjects who are part of a previously poorly characterized population. Africans represent the most genetically diverse population in the world, but have not been as well studied with respect to their HLA types compared to the inhabitants of western countries. The presently disclosed high-throughput HLA sequencing technology was applied to a cohort of 402 healthy adolescents from Cape Town, South Africa, as part of a study of T-cell responses to Mycobacterium tuberculosis.

### Methods

Previously cryopreserved PBMC samples collected from 403 adolescents were processed and high resolution HLA typing was performed using a MIA FORA NGS kit and analysis software. MIA FORA NGS was designed specifically for HLA typing to provide accurate, comprehensive coverage of all major HLA gene regions, including whole gene coverage for HLA-A, -B, -C, -DPA1, -DQA1, and -DQB1; all exons and introns for -DRB1 and -DRB3/4/5 except partial coverage for exon 6 and intron 1; and all exons and introns between exons 2 and 4 for HLA-DPB1. NGS sequencing libraries were prepared using a semi-automated protocol and sequenced in high-throughput format (384 samples per run) on the Illumina NextSeq platform. HLA allele candidates were computed and final HLA typing was confirmed using MIA FORA NGS analysis software. High resolution HLA typing of 11 HLA loci revealed a unique population, including unusual haplotypes, and approximately 30 novel alleles not previously reported in the IMGT database.

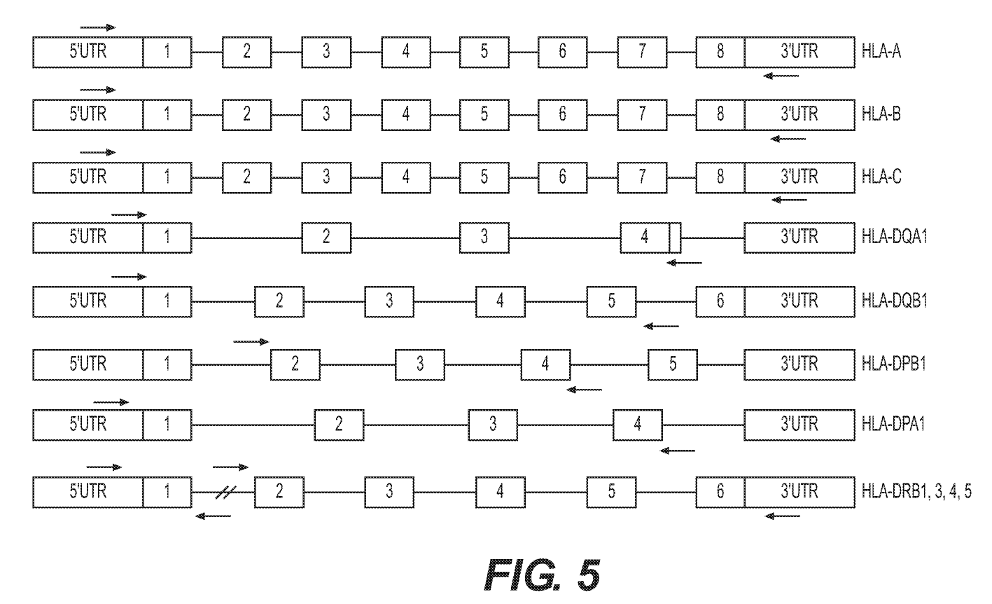

Each HLA gene was amplified from genomic DNA using a single long-range PCR reaction. As shown in FIG. 5, human genomic DNA was amplified by long-range PCR, targeting 11 HLA genes in nine all-in-one master mixes. Whole gene coverage included HLA-A, -B, -C, -DPA1, -DQA1, and -DQB1; all exons and introns for -DRB1 and -DRB3/4/5 except partial coverage for exon 6 and intron 1; and all exons and introns between exons 2 and 4 for HLA-DPB1. Following amplification, PCR products were measured and balanced, then fragmented, repaired and ligated to index adaptors containing unique barcodes. Then, the barcoded samples were consolidated into a single sequencing library, size selected, and amplified a few rounds to incorporate the P5 and P7 adaptor sequences needed for binding to the Illumina flow cell. Samples were processed in sets of 96 samples, combined in a sequencing library with up to 384 samples and sequenced on an Illumina NextSeq instrument. The semi-automated workflow was facilitated by using Biomek liquid handlers.

Raw NGS sequence reads were used as input for the MIA FORA NGS software to sort index adaptors from different samples and to generate accurate genotype calls for all 11 major HLA genes with high resolution. Three orthogonal algorithms were combined to rank the genotype candidates and generate consensus sequences for individual alleles. HLA genotypes called automatically by the software have been validated across multiple studies and shown to have a concordance greater than 97%. In the same studies, manual review of called genotypes increased concordance to greater than 99%. Haplotype analyses were performed using the computer package PYPOP (ref). Allele frequencies were obtained by direct counting, assuming no blank frequencies.

### Novel Alleles

High resolution genotyping enabled discovery of novel HLA variants. In this study 12 different variants were observed in two or more samples. They include single base substitutions in Class I and Class II loci, and variant alleles that may have resulted from recombination or exon rearrangements. All are listed in Table 2.

TABLE 2
List of Common Novel Alleles in Cape Town Cohort

| | N | |
|---|---|---|
| Provisional Name | Alleles | Sequence Details |
| A*32:01:01x1 | 8 | Novel A*32:01:01 (exon 1) 67 T > A |
| A*32:01:01x2 | 3 | Novel A*32:01:01 (exon 5) AGG > |
| | | AGA R307R |
| C*17:01:01:02x1 | 2 | Novel C*17:01:01:02 (exon 4) |
| | | CCC > CTC P193L |
| DPA1*02:01:01x1 | 9 | Novel DPA1*02:01:01 (exon 2) |
| | | CGT > TGT R73C |
| DPA1*02:02:01x1 | 15 | DPA1*02:02:01 novel exon 3 |
| DPA1*03:01x1 | 6 | Novel DPA1*03:01 (exon 4) GTG > |
| | | GTC V201V |
| DPA1*04:01x1 | 13 | Novel DPA1*04:01 (exon 4) |
| | | GCG > ACG A187T |
| DPB1*11:01:01x1 | 7 | Novel DPB1*11:01:01 (exon 4) |
| | | CGG > CAG R189Q |
| DPB1*106:01x1 | 2 | Novel DPB1*106:01 (exon 2) |
| | | AGG > GGG R70G |
| DQA1*04:01:01x1 | 2 | Novel DQA1*04:01:01 (exon 2) |
| DQB1*02:02:01:01v1 | 12 | Novel intron DQB1*02:02:01:01: intron |
| | | matches DQB1*02:01:01 |
| DQB1*05:01:01:01x1 | 2 | Novel DQB1*05:01:01:01 (exon 1) |
| | | TCC > TCT S(−10)S |

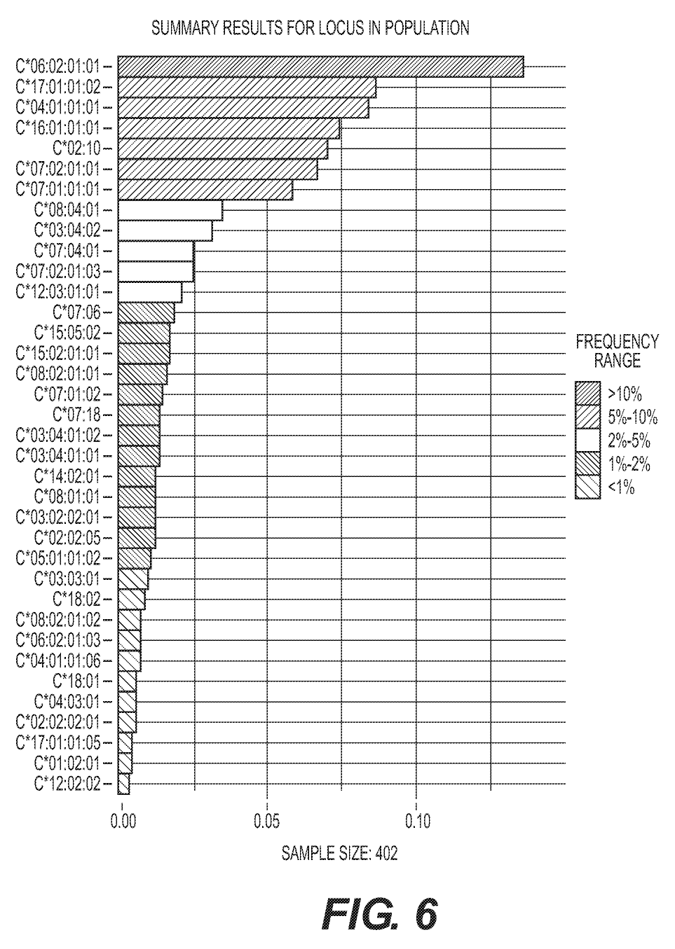

The Cape Town cohort had 58 different alleles of HLA-A, 71 of HLA-B, and 47 of HLA-C. Of these, 21 alleles of HLA-A, 26 of HLA-B and 18 of HLA-C were found at a high global frequency. Comparisons were also made with previous data from Africa, including five African populations (Cao et al.) and African Americans in the US population (Maiers et al.) In general, the alleles found at high frequency in Cape Town were similar to other African populations but there were exceptions. For example, there were five HLA-B (frequency of 0.044), three HLA-A and two HLA-C alleles that are more common in Asia. The frequency of the top 15 Cape Town alleles was 0.68 in HLA-A compared to 0.65 in HLA-B and 0.77 in HLA-C; the values for African Americans were higher, namely 0.85 for HLA-A, 0.73 for HLA-B and 0.94 for HLA-C. FIG. 6 shows the allele distribution for HLA-C in the cohort.

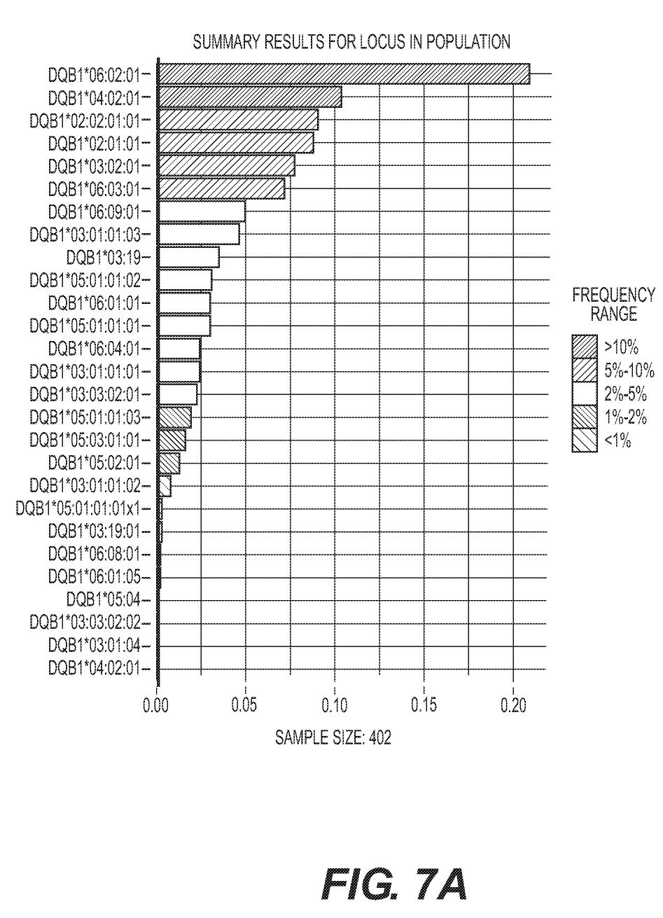

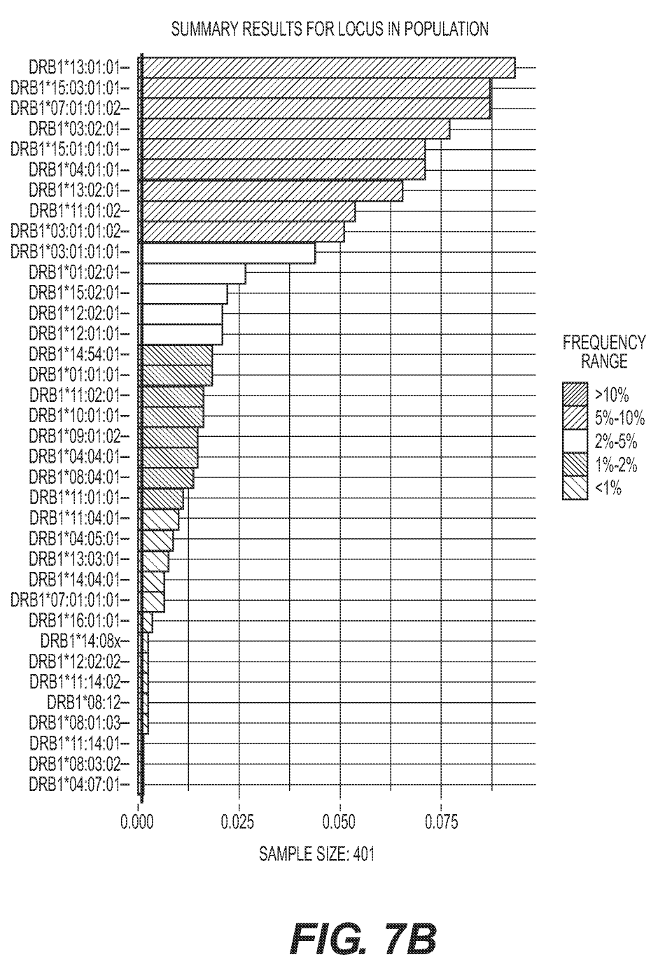

FIG. 7A shows the allele distribution for HLA-DQB, while FIG. 7B shows the allele distribution for HLA-DRB in the cohort. There were 31 A-B-DRB1 haplotypes found in 4 or more Cape Town samples, listed in Table 3. Half of the haplotypes were found in the US donor population and half were not. All except three (28 haplotypes) were found in the African American cohort, compared to only nine in the Europeans and 13 in the Hispanic group. The most frequent haplotype in African Americans (A*30:01:01-B*42:01:01-DRB1*03:02:01) was also the most frequent in Cape Town. There were only five haplotypes in common with the Asian Pacific Islander cohort but one of them was the third most common haplotype (A*33:03:01-B*44:03:02-DRB1*07:01:01:02).

TABLE 3
Cape Town A---B---DRB1 Haplotypes Sorted by Frequency

| A-B-DRB1 Haplotypes | Frequency |
|---|---|
| A*30:01:01:B*42:01:01:DRB1*03:02:01 | 0.03117 |
| A*02:01:01:01:B*45:01:01:DRB1*13:01:01 | 0.01621 |
| A*68:02:01:01:B*15:10:01:DRB1*03:01:01:02 | 0.01231 |
| A*03:01:01:01:B*07:02:01:DRB1*15:01:01:01 | 0.00998 |
| A*23:01:01:B*15:10:01:DRB1*11:01:02 | 0.00873 |
| A*74:01:01:B*15:03:01:02:DRB1*13:02:01 | 0.00748 |
| A*68:01:01:02:B*58:02:DRB1*07:01:01:02 | 0.00748 |
| A*29:01:01:01:B*18:01:01:02:DRB1*13:02:01 | 0.00748 |
| A*29:02:01:01:B*44:03:02:DRB1*11:01:02 | 0.00748 |
| A*30:04:01:B*39:10:01:DRB1*15:03:01:01 | 0.00748 |
| A*43:01:B*15:10:01:DRB1*04:01:01 | 0.00623 |
| A*03:01:01:05:B*08:01:01:DRB1*03:02:01 | 0.00623 |
| A*02:01:01:01:B*45:07:DRB1*13:01:01 | 0.00623 |
| A*02:05:01:B*14:01:01:DRB1*13:01:01 | 0.00623 |
| A*02:01:01:01:B*07:02:01:DRB1*15:01:01:01 | 0.00623 |
| A*02:05:01:B*58:01:01:01:DRB1*11:02:01 | 0.00623 |
| A*66:02:B*42:01:01:DRB1*03:02:01 | 0.00623 |
| A*24:02:01:01:B*57:01:01:DRB1*07:01:01:02 | 0.00499 |
| A*02:01:01:01:B*40:01:02:DRB1*13:02:01 | 0.00499 |
| A*29:02:01:02:B*42:01:01:DRB1*03:02:01 | 0.00499 |
| A*30:02:01:02:B*45:01:01:DRB1*01:02:01 | 0.00499 |
| A*23:17:B*39:10:01:DRB1*15:03:01:01 | 0.00499 |
| A*24:02:01:01:B*07:02:01:DRB1*15:03:01:01 | 0.00499 |
| A*66:01:01:B*58:02:DRB1*13:01:01 | 0.00499 |
| A*30:01:01:B*07:02:01:DRB1*01:02:01 | 0.00499 |
| A*43:01:B*44:03:01:01:DRB1*04:01:01 | 0.00499 |
| A*33:03:01:B*44:03:02:DRB1*07:01:01:02 | 0.00499 |
| A*24:07:01:B*35:05:01:DRB1*12:02:01 | 0.00499 |
| A*30:02:01:02:B*58:02:DRB1*15:03:01:01 | 0.00499 |
| A*02:02:01:01:B*57:03:01:DRB1*01:02:01 | 0.00499 |
| A*30:01:02:B*81:01:DRB1*15:03:01:01 | 0.00499 |

Two locus haplotypes in the Cape Town cohort included 341 for A-B, 302 for A-C and 167 for B-C. The top five haplotypes of each were found at a frequency of 0.108, 0.133, and 0.236 respectively (Table 3). Linkage disequilibrium was moderate for A-B and A-C haplotypes (D′ 0.334-0.722) but was stronger for B-C (D′ 0.628-1.0) reflecting the closer genetic distance between those loci. The top four AB haplotypes were also found in at least three of the African populations reported by Cao et al, and were the highest frequency haplotypes in African Americans of the US population reported by Maiers et al. The fifth haplotype was not observed in any African group (Cao et al.); however, it was the second most common haplotype in the US population with a five-fold higher frequency in donors with European ancestry (Maier et al.) The top four B-C haplotypes were also reported by Cao et al. but the fifth most frequent haplotype (B*15:03C*02:10) was not previously reported, most likely because C*02:10 was not differentiated from C*02:01 in the previous studies.

TABLE 4
Top Five Two Locus Haplotypes Observed in Cape Town

| Locus | Top Five Allele Pairs | Freq. | Count | Ld. D'ij | Total Freq. |
|---|---|---|---|---|---|
| A~B | A*30:01:01~B*42:01:01 | 0.03549 | 28.5 | 0.52581 | 0.108 |
| (341 total) | A*68:02:01:01~B*15:10:01 | 0.02162 | 17.4 | 0.38502 | |
| | A*02:01:01:01~B*45:01:01 | 0.02027 | 16.3 | 0.39556 | |
| | A*68:01:01:02~B*58:02 | 0.01617 | 13 | 0.58833 | |
| | A*03:01:01:01~B*07:02:01 | 0.01493 | 12 | 0.33856 | |
| A~C | A*30:01:01~C*17:01:01:02 | 0.04550 | 36.6 | 0.60768 | 0.133 |
| (302 total) | A*02:01:01:01~C*16:01:01:01 | 0.02961 | 23.8 | 0.33384 | |
| | A*68:01:01:02~C*06:02:01:01 | 0.01987 | 16 | 0.72291 | |
| | A*68:02:01:01~C*03:04:02 | 0.01965 | 15.8 | 0.61218 | |
| | A*01:01:01:01~C*07:01:01:01 | 0.01836 | 14.8 | 0.36162 | |
| B~C | B*58:02~C*06:02:01:01 | 0.06838 | 55 | 0.90304 | 0.236 |
| (167 total) | B*42:01:01~C*17:01:01:02 | 0.06343 | 51 | 1 | |
| | B*08:01:01~C*07:01:01:01 | 0.03799 | 30.5 | 0.62817 | |
| | B*45:01:01~C*16:01:01:01 | 0.03358 | 27 | 0.72984 | |
| | B*15:03:01:02~C*02:10 | 0.03234 | 26 | 1 | |
| DQA1~DRB1 | DQA1*01:03:01:02~DRB1*13:01:01 | 0.08727 | 70 | 0.91256 | 0.362 |
| (101 total) | DQA1*04:01:01~DRB1*03:02:01 | 0.07606 | 61 | 0.96536 | |
| | DQA1*03:03:01~DRB1*04:01:01 | 0.06983 | 56 | 0.95992 | |
| | DQA1*01:02:01:03~DRB1*15:03:01:01 | 0.06815 | 54.7 | 0.72848 | |
| | DQA1*01:02:01:04~DRB1*13:02:01 | 0.06108 | 49 | 0.97832 | |
| DQA1~DQB1 | DQA1*01:02:01:03~DQB1*06:02:01 | 0.13714 | 110.3 | 0.87813 | 0.403 |
| (78 total) | DQA1*04:01:01~DQB1*04:02:01 | 0.08085 | 65 | 0.96671 | |
| | DQA1*03:03:01~DQB1*03:02:01 | 0.0709 | 57 | 0.90603 | |
| | DQA1*01:03:01:02~DQB1*06:03:01 | 0.06581 | 52.9 | 0.90303 | |
| | DQA1*05:01:01:02~DQB1*02:01:01 | 0.04851 | 39 | 1 | |
| DPA1~DQB1 | DPA1*01:03:01:02~DQB1*06:02:01 | 0.05091 | 40.9 | 0.0817 | 0.200 |
| (162 total) | DPA1*02:02:02~DQB1*06:02:01 | 0.04371 | 35.1 | 0.0137 | |
| | DPA1*02:02:02~DQB1*04:02:01 | 0.04133 | 33.2 | 0.25257 | |
| | DPA1*02:02:02~DQB1*03:02:01 | 0.03731 | 30 | 0.35655 | |
| | DPA1*02:01:01~DQB1*06:02:01 | 0.02689 | 21.6 | 0.02821 | |
| DPA1~DPB1 | DPA1*02:02:02~DPB1*01:01:01 | 0.14558 | 116.8 | 0.66017 | 0.491 |
| (96 total) | DPA1*03:01~DPB1*105:01 | 0.11222 | 90 | 0.91204 | |
| | DPA1*01:03:01:01~DPB1*02:01:02 | 0.09975 | 80 | 0.90899 | |
| | DPA1*01:03:01:02~DPB1*04:01:01:01 | 0.08552 | 68.6 | 0.50511 | |
| | DPA1*01:03:01:04~DPB1*04:01:01:01 | 0.04778 | 38.3 | 0.8977 | |

High resolution HLA typing is a powerful method to characterize HLA allele and haplotype diversity in population studies. Whole gene coverage provided extensive polymorphic sites that define the physical linkage between exons and helps to resolve trans, or combination ambiguities in phasing. HLA typing to single nucleotide resolution allowed detection of previously unreported variants, including coding sequence changes.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present disclosure. The disclosure is intended to cover the components and steps in any sequence which is effective to meet the objectives there intended, unless the context specifically indicates the contrary.

### REFERENCES

1) Cao, K., et al. 2004. Differentiation between African populations is evidenced by the diversity of alleles and haplotypes of HLA class I loci. Tissue Antigens 63:293-325.
2) Lancaster A, Nelson M P, Meyer D, Single R M, Thomson G. 2003. PyPop: a software framework for population genomics: analyzing large-scale multi-locus genotype data. Pac Symp Biocomput. 2003:514-525.
3) Maiers, M., Gragert, L., Klitz, W. High resolution HLA alleles and haplotypes in the US population. 2007. Human Immunology 68:779-788.
4) O'Garra, Anne, et al. 2013. Annual Review of Immunology 31:475-527.
5) Single R M, Meyer D, Mack S J, Lancaster A, Erlich H A, Thomson G. 2007. 14th International HLA and Immunogenetics Workshop: report of progress in methodology, data collection, and analyses. Tissue Antigens 69 Suppl 1:185-187.
6) Wang, C., Krishnakumar, S., Wilhelmy, J., Babrzadeh, F., Stepanyan, L., Su, L. F., Levinson, D., Fernandez-Viva, M., Davis, R. W., Davis, M. M., Mindrinos, M. 2012. High-throughput, high-fidelity HLA genotyping with deep sequencing. Proc. Natl. Acad. Sci. 109(22):8676-8681.
7) Wang C, et al. (2009) High-throughput, high-fidelity HLA genotyping with deep sequencing. PNAS 109(22):8676-8681.

1. A method for determining the association of Human Leukocyte Antigen (HLA) alleles at adjacent loci in genomic DNA from a biological sample obtained from a human subject, comprising:
a. amplifying genomic DNA from the sample by long-range PCR reaction;
b. sequencing the amplified DNA;
c. determining the association frequency of the genotype of an allele of a first locus with the genotype of an allele of at least one adjacent locus by reference to a database of loci associations; and
d. reporting an association score of said allele in a first locus with the genotype of the allele in at least one adjacent locus, thereby determining an association of Human Leukocyte Antigen (HLA) alleles at adjacent loci in genomic DNA.

2. The method of claim 1, wherein said association is determined between three or more loci.

3-5. (canceled)

6. The method of claim 1, wherein said first allele is an allele of an HLA locus selected from the group consisting of HLA-A, HLA-DRB1, HLA-DRB3, HLA-DRB4, HLA-DRB5, HLA-DQB1, HLA-DQA1, HLA-DPB1 and HLA-DPA1.

7. The method of claim 1, wherein said database of associations comprises associations of HLA loci for at least 2 loci selected from: HLA-A, HLA-DRB1, HLA-DRB3, HLA-DRB4, HLA-DRB5, HLA-DQB1, HLA-DQA1, HLA-DPB1 and HLA-DPA1.

8. The method of claim 1, wherein the at least one adjacent locus is in linkage disequilibrium with the first locus.

9. The method of claim 2, wherein at least one of said two or more loci are in linkage disequilibrium with said first locus.

10-12. (canceled)

13. The method of claim 1, further comprising comparing the association score to a database of association scores associated with a disease.

14. (canceled)

15. The method of claim 1, further comprising comparing the association score to an association score obtained for a different human subject for assessing tissue compatibility.

16-19. (canceled)

20. A method of operating a computing device comprising at least one processor, the method comprising:
executing the at least one processor to acquire genotype information representing an assignment of an allele variant to a genetic human leukocyte antigen (HLA) locus; and
when it is determined that a score representing an association of the allele variant with at least one other allele variant of at least one adjacent genetic locus exists:
using the determined score to generate an indication indicating the likelihood of the correctness of the assignment of the allele variant to the genetic HLA locus; and reporting the indication.

21-26. (canceled)

27. The method of claim 20, further comprising executing the at least one processor to determine whether the score exists by computing with information, by the at least one processor, in a linkage disequilibrium database.

28. The method of claim 27, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRBS, HLA-DQB1, HLA-DQA1, HLA-DPB1 and HLA-DPA1, where HLA-DRB3, HLA-DRB4 and HLA-DRBS together are considered one locus.

29-44. (canceled)

45. A system for validating correctness of a genotype of a sample obtained from a subject, the system comprising:
at least one processor;
a memory communicatively coupled to the processor, the memory having stored thereon computer executable instructions that, when executed by the at least processor, perform a method comprising:
acquiring genotype information representing an assignment of an allele variant to a genetic human leukocyte antigen (HLA) locus;
determining a score representing an association of the allele variant with at least one other allele variant of at least one adjacent genetic locus;
using the determined score to generate an indication indicating correctness of the assignment of the allele variant to the genetic HLA locus; and
reporting the indication.

46-48. (canceled)

49. The system of claim 45, wherein determining the score comprises accessing a linkage disequilibrium database.

50. The system of claim 49, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRBS, HLA-DQB1, HLA-DQA1, HLA-DPB1 and HLA-DPA1, where HLA-DRB3, HLA-DRB4 and HLA-DRB5 together are considered one locus.

51-55. (canceled)

56. At least one non-transitory computer storage device storing computer-executable instructions that, when executed by at least one processor, cause the at least one processor to perform a method comprising:
acquiring a genotype information representing an assignment of an allele variant to a genetic human leucocyte antigen (HLA) locus;
acquiring a score representing an association of the allele variant with at least one other allele variant of at least one adjacent genetic locus;
using the acquired score to generate an indication indicating correctness of the assignment of the allele variant to the genetic HLA locus; and
reporting the indication.

57-59. (canceled)

60. The at least one non-transitory computer storage device of claim 56, wherein determining the score comprises accessing a linkage disequilibrium database.

61. The at least one non-transitory computer storage device of claim 60, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRBS, HLA-DQB1, HLA-DQA1, HLA-DPB1 and HLA-DPA1, where HLA-DRB3, HLA-DRB4 and HLA-DRBS together are considered one locus.

62-69. (canceled)

70. A method of assigning an allele to a genetic locus comprising:
amplifying coding and non-coding DNA from a genetic locus from a sample of genomic DNA to produce an amplicon sequencing the amplicon;
identifying at least a first allele variant and a second allele variant of the genetic locus from the amplicon;
determining a score representing an association of the first allele variant with at least one other allele variant of at least one adjacent genetic locus; and
using the determined score to generate an indication indicating correctness of the assignment of the allele variant to the genetic HLA locus.

71-85. (canceled)

86. The method of claim 70, wherein determining the score comprises accessing a linkage disequilibrium database.

87. The method of claim 86, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRBS, HLA-DQB1, HLA-DQA1, HLA-DPB1 and HLA-DPA1, where HLA-DRB3, HLA-DRB4 and HLA-DRBS together are considered one locus.

88-92. (canceled)


SEQUENCE LISTING (12 sequences)

SEQ ID NO: 1 - DNA, 125 bp, Artificial Sequence
  Description of Artificial Sequence Synthetic polynucleotide
ggctctcactccatgaggtatttcttcacatccgtgtcccggcccggccgcggggagccccgcttcatcgcagtgggctacgtggacgacacgcagttcgtgcggttcgacagcgacgccgcgag

SEQ ID NO: 2 - DNA, 125 bp, Artificial Sequence
  Description of Artificial Sequence Synthetic polynucleotide
ggctcccactccatgaggtatttctccacatccgtgtcccggcccggccgcggggagccccgcttcatcgccgtgggctacgtggacgacacgcagttcgtgcggttcgacagcgacgccgcgag

SEQ ID NO: 3 - DNA, 58 bp, Artificial Sequence
  Description of Artificial Sequence Synthetic oligonucleotide
ggctcccactccatgaggtatttctccacatccgtgtcccggcccggccgcggggagc

SEQ ID NO: 4 - DNA, 66 bp, Artificial Sequence
  Description of Artificial Sequence Synthetic oligonucleotide
ccgcttcatcgcagtgggctacgtggacgacacgcagttcgtgcggttcgacagcgacgccgcgag

SEQ ID NO: 5 - DNA, 100 bp, Artificial Sequence
  Description of Artificial Sequence Synthetic polynucleotide
catgaggtatttcttcacatccgtgtcccggcccggccgcggggagccccgcttcatcgcagtgggctacgtggacgacacgcagttcgtgcggttcgac

SEQ ID NO: 6 - DNA, 13 bp, Artificial Sequence
  Description of Artificial Sequence Synthetic oligonucleotide
gcgacgccgcgag

SEQ ID NO: 7 - DNA, 52 bp, Artificial Sequence
  Description of Artificial Sequence Synthetic oligonucleotide
ggctctcactccatgaggtatttcttcacatccgtgtcccggcccggccgcg

SEQ ID NO: 8 - DNA, 68 bp, Artificial Sequence
  Description of Artificial Sequence Synthetic oligonucleotide
ccccgcttcatcgcagtgggctacgtggacgacacgcagttcgtgcggttcgacagcgacgccgcgag

SEQ ID NO: 9 - DNA, 375 bp, Artificial Sequence
  Description of Artificial Sequence Synthetic polynucleotide
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtgggtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctg

SEQ ID NO: 10 - DNA, 375 bp, Artificial Sequence; identical to SEQ ID NO: 9
  Description of Artificial Sequence Synthetic polynucleotide

SEQ ID NO: 11 - DNA, 375 bp, Artificial Sequence
  Description of Artificial Sequence Synthetic polynucleotide
  modified_base (1)..(73): a, c, t, g, unknown or other
nnnnnnnnnnnnnnnnnnnnnnnnnnnnnnnnnnnnnnnnnnnnnnnnnnnnnnnnnnnnnnnnnnnnnnnnngctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtgggtggcggagcagttgagagcctacctggagggcacgtgcgtggagtggctccgcagatacctggagaacgggaaggagacgctgcagcgcacggatgcccccaaaacgcatatgactcaccacgctgtctctgaccatgaagccaccctg

SEQ ID NO: 12 - DNA, 375 bp, Artificial Sequence
  Description of Artificial Sequence Synthetic polynucleotide
  modified_base (1)..(73): a, c, t, g, unknown or other
nnnnnnnnnnnnnnnnnnnnnnnnnnnnnnnnnnnnnnnnnnnnnnnnnnnnnnnnnnnnnnnnnnnnnnnnngctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtgggtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctg